(19) United States
(12) Patent Application Publication
(10) Pub. No.: US 2026/0248463 A1
(43) Pub. Date: Aug. 27, 2026

(54) PERSONAL CARE DEVICE AND RESCUE MONITORING AND RELIGIOUS MUSIC PLAYBACK METHOD

(71) Applicants: Jian-Hung LIOU, Yunlin County (TW); Chin-Hao CHANG, New Taipei City (TW); Shao-Wen CHANG, Taipei City (TW); Cheng-Ching CHIANG, New Taipei City (TW)

(72) Inventors: Jian-Hung LIOU, Yunlin County (TW); Chin-Hao CHANG, New Taipei City (TW); Shao-Wen CHANG, Taipei City (TW)

(21) Appl. No.: 19/538,936

(22) Filed: Feb. 12, 2026

(30) Foreign Application Priority Data
Feb. 26, 2025 (TW) 114107202

(51) Int. Cl. A61B 5/00

(52) U.S. Cl. CPC A61B 5/743

(57) ABSTRACT

A personal care device id disclosed to detect activity signals of a user within a living environment, generate an initial fall signal when determining that the user has fallen, trigger a user fall detection alarm based on the initial fall signal, generate a first infrared thermal imaging signal associated with a daily living activity of the user, analyze the first infrared thermal imaging signal based on the initial fall signal to generate a vital sign signal of the user, analyze the vital sign signal of the user to determine a current physiological state of the user, and trigger an emergency medical alert and t play a religious music within the living environment of the user for the user to listen to when determining that a vital sign of the user is outside a safety range.

### BACKGROUND OF THE DISCLOSURE

### Technical Field

The present disclosure relates to a care device and a monitoring method, particularly to a personal care device and a rescue monitoring and religious music playback method.

### Description of Related Art

Currently, an elderly fall detection system typically utilizes sensors or image processing techniques to determine a fall status and transmit an alarm to a user. However, due to limitations in the detection performance or detection range of the sensors, there is a possibility of false determination, which may reduce the user's confidence. Furthermore, the functionality of the sensors is limited to fall detection and does not provide further support or comfort in the physiological or psychological aspects. In addition, elderly individuals living alone may not receive timely rescue after a fall, a situation that is particularly severe in remote areas, and may even lead to tragic events such as “death by loneliness” because the elderly are not discovered for a long time.

In societies with aging populations, how to provide comprehensive personal care encompassing both physiological and psychological well-being is an issue that requires careful consideration.

### SUMMARY OF THE DISCLOSURE

One embodiment of the present disclosure provides a personal care device including a fall detection module, a fall data processing module, an alarm module, a first infrared thermal imaging detection module, a physiological information processing module, a vital detection module, and a religious music playback module. The fall detection module is configured to detect one or more types of activity signals of a daily living activity of a user within a living environment. The fall data processing module is configured to analyze and process the one or more types of activity signals and to generate an initial fall signal when the fall data processing module determines that the user has fallen. The alarm module is coupled to the fall data processing module and configured to trigger a user fall detection alarm based on the initial fall signal. The first infrared thermal imaging detection module is configured to generate a first infrared thermal imaging signal associated with the daily living activity of the user. The physiological information processing module is coupled to the first infrared thermal imaging detection module and the fall data processing module, and configured to analyze the first infrared thermal imaging signal based on the initial fall signal to generate a vital sign signal of the user. The vital detection module is coupled to the physiological information processing module and the alarm module, and configured to analyze the vital sign signal of the user to determine a current physiological state of the user. The religious music playback module is coupled to the vital detection module, wherein the vital detection module is configured to determine when a vital sign of the user is outside a safety range, the alarm module is configured to trigger an emergency medical alert, and the religious music playback module is configured to play a religious music within the living environment of the user for the user to listen to.

Another embodiment of the present disclosure provides a rescue monitoring and religious music playback method, adapted for execution by a personal care device, wherein the personal care device includes a fall detection module, a fall data processing module, an alarm module, a physiological information processing module, a vital detection module, and a religious music playback module, and the rescue monitoring and religious music playback method includes: detecting one or more types of activity signals of a daily living activity of a user within a living environment through the fall detection module; analyzing and processing the one or more types of activity signals and generating an initial fall signal when the fall data processing module determines that the user has fallen; triggering a user fall detection alarm based on the initial fall signal through the alarm module; receiving the initial fall signal and analyzing a first infrared thermal imaging signal generated by a first infrared thermal imaging detection module to generate a vital sign signal of the user through the physiological information processing module; analyzing the vital sign signal of the user to determine a current physiological state of the user through the vital detection module; and when the vital detection module determines that a vital sign of the user is not within a safety range, triggering an emergency medical alert through the alarm module, and playing religious music in the living environment of the user for the user to listen to through the religious music playback module.

### BRIEF DESCRIPTION OF THE DRAWINGS

FIG. 1 is a block diagram of the personal care device in accordance with the present disclosure.

FIG. 2 is a block diagram of the personal care device in accordance with another embodiment of the present disclosure.

FIG. 3 is a block diagram of the personal care device in accordance with still another embodiment of the present disclosure.

FIG. 4 is a flowchart of the rescue monitoring and religious music playback method in accordance with the present disclosure.

### DETAILED DESCRIPTION

The following description, taken in conjunction with the accompanying drawings and embodiments, is provided to further illustrate the present disclosure to enable a person having ordinary skill in the art to better understand and practice the present disclosure. However, the specific embodiments described herein are not intended to limit the scope of the present disclosure.

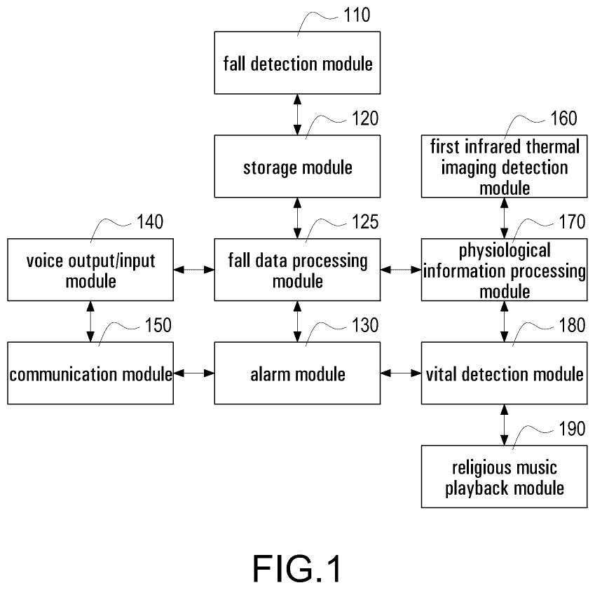

FIG. 1 is a block diagram of the personal care device in accordance with the present disclosure.

A personal care device may be disposed at a fixed location within a living environment of a user, such as a living room, bathroom, kitchen, bedroom, or garden; alternatively, the personal care device may be disposed at an electronic device capable of free movement within the living environment, such as a robot. Whether disposed at a fixed location or as a freely movable robot, the personal care device of the present disclosure may implement a rescue monitoring and religious music playback method described herein.

Based on medical rescue experience, a physical condition of elderly individuals is typically normal; however, after an initial fall, the physical condition may rapidly deteriorate, in addition to any injuries sustained from the fall. In other words, an accidental fall of elderly individuals is a significant indicator of a decline in the physical condition. Therefore, the personal care device monitors the user to detect whether an accidental fall event has occurred. For example, the personal care device of the present disclosure may utilize wide-range or high-resolution infrared thermal imaging to determine whether a body temperature of the user is within a normal range. If the body temperature of the user is outside the normal range, it may indicate an accidental fall and hypothermia of the user. The personal care device of the present disclosure may also utilize millimeter wave detection to assess the likelihood of a fall based on a movement distance of the user (e.g., a significant movement or displacement within milliseconds). Such significant movement or displacement may indicate an accidental fall. Furthermore, the personal care device of the present disclosure may utilize image detection to analyze image features and determine whether the user has experienced an accidental fall. By analyzing movement features of the user through image analysis, it may be directly determined whether an accidental fall may have occurred. When a potential accidental fall event occurs, the personal care device issues a first-stage alert. However, the aforementioned scenarios may result in a false determination; to address the false determination, after detecting a potential accidental fall event, the personal care device of the present disclosure further utilizes a small-range or low-resolution infrared thermal imaging to further confirm the body temperature of the user, or utilizes voiceprint recognition to perform a rapid background assessment of the user's condition, such as detecting groans or cries. Because a small-range or low-resolution infrared thermal imaging may quickly obtain the body temperature values of the user (without requiring a high-precision temperature distribution or range), and voiceprint recognition may quickly compare the meaning represented by the sounds produced by the user, a secondary confirmation may be performed relatively quickly to eliminate the false determination. After the secondary confirmation, if the user indeed requires assistance, the personal care device issues a second alert, notifying medical personnel or family members to quickly arrive at the scene to assist the user. In addition, before medical personnel or family members arrive at the scene and the user is nearing the end of life (referring to the assessment mechanism for signs of imminent death in medical care), the personal care device of the present disclosure plays the religious music to ensure that the user neither feels isolated nearing the end of life, nor experiences fear of death, thereby allowing them to peacefully alleviate suffering and transition to the next life.

The personal care device of the present disclosure includes two detection and alert stages. A first fall detection alert is triggered when the personal care device initially detects that the user has fallen. To avoid the false determination of the first fall detection alert, which could lead to unnecessary and erroneous notifications, the personal care device further confirms the user's status to determine whether to trigger a second fall detection alert. Details are described below.

As shown in FIG. 1, the personal care device includes a fall detection module 110, a storage module 120, a fall data processing module 125, an alarm module 130, a voice output/input module 140, a communication module 150, a first infrared thermal imaging detection module 160, a physiological information processing module 170, a vital detection module 180, and a religious music playback module 190.

The fall detection module 110 is coupled to the storage module 120, and is configured to detect one or more types of activity signals of a daily living activity of a user in the living environment. The activity signal detected by the fall detection module 110 is stored in the storage module 120 and is accessible by the fall data processing module 125, which analyzes the activity signal to determine whether the user has fallen and generates an initial fall signal, serving as a first fall detection alert.

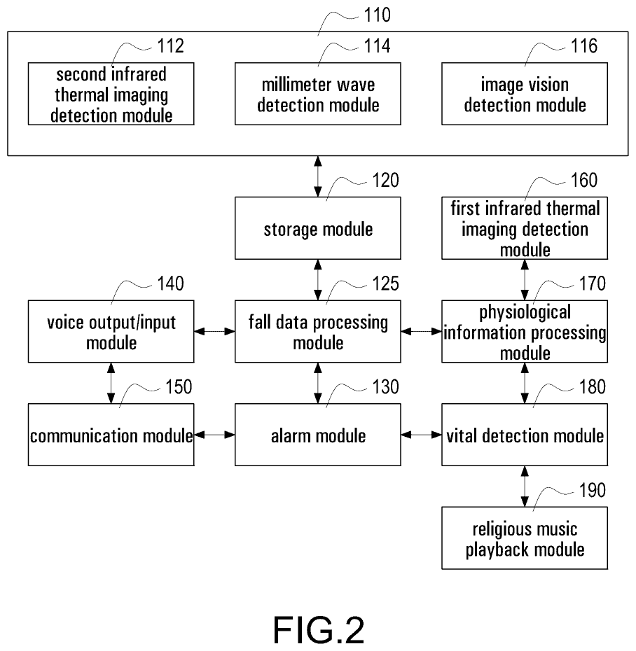

The fall data processing module 125 is coupled to the storage module 120, the alarm module 130, the voice output/input module 140, and the physiological information processing module 170. The fall data processing module 125 reads the one or more types of activity signals from the storage module 120, and analyzes and processes the activity signal to determine a current physical or mental condition of the user, such as whether a fall has occurred. The types of activity signals are described in detail in FIG. 2.

### First Embodiment: The First Fall Detection Alert

The fall data processing module 125 uses the activity signal stored in the storage module 120 to determine whether the user has fallen. If the fall data processing module 125 preliminarily determines that the user may have fallen, an initial fall signal is generated. At this time, the alarm module 130 triggers a user fall detection alarm based on the initial fall signal, the voice output/input module 140 receives a voice signal from the user, and the communication module 150 transmits a user on-site voice signal and the user fall detection alarm to an electronic device (not shown) or a remote control center (not shown). After the electronic device or the remote control center receives the user fall detection alarm, a monitor actively sends a query to confirm the user's condition. The monitor's voice signal is played back via the communication module 150 and the voice output/input module 140, allowing the monitor to confirm whether the user has fallen or experienced another incident.

If the user has not experienced a fall, the user responds to the monitor via the voice output/input module 140, reporting that they are well, thereby reassuring the monitor. If the user has indeed experienced a fall and is still conscious and experiencing pain, the user responds to the monitor via the voice output/input module 140, describing their current condition, in order to seek assistance from the monitor.

### Second Embodiment: Second Detection and Alert Stage

The first infrared thermal imaging detection module 160 is coupled to the physiological information processing module 170. The vital detection module 180 is coupled to the physiological information processing module 170 and the religious music playback module 190.

The first infrared thermal imaging detection module 160 is configured to generate a first infrared thermal imaging signal. The first infrared thermal imaging detection module 160 is a low-angle (e.g., less than or equal to 60 degrees) and low-resolution (e.g., less than or equal to 60×80 pixels, such as 8×8 pixels, 16×16 pixels, or 32×32 pixels) infrared thermal imaging sensor, used to sense images with a smaller viewing angle or lower resolution.

The first infrared thermal imaging signal includes temperature, breathing, and heartbeat or heart rhythm, and represents a signal with a smaller viewing angle or lower resolution. The temperature, breathing, and heartbeat or heart rhythm may be used to determine the physical condition of the user, such as whether the body temperature is within a normal range, whether the breathing is within a normal frequency range, or whether the heartbeat or heart rhythm is within a normal frequency range.

During the first detection and alert stage, the fall data processing module 125 detects that the user has fallen and generates an initial fall signal to the physiological information processing module 170. At this time, the second detection and alert stage is entered, and the physiological information processing module 170, upon receiving the initial fall signal, is triggered and begins to analyze the first infrared thermal imaging signal to generate a vital sign signal of the user, such as body temperature, breathing, and heartbeat or heart rhythm. The physiological information processing module 170 transmits an analyzed vital sign signal to the vital detection module 180.

The vital detection module 180 analyzes the vital sign signal of the user to determine a current physiological state of the user, such as the degree of the vital sign (e.g., whether the breathing, pulse, or body temperature is within a range requiring medical or emergency intervention). The vital detection module 180 analyzes a real-time physiological state of the user to determine the normal or weakened level of the vital sign of the user, which serves as a basis for the alarm module 130 to trigger an emergency medical alert.

If the vital detection module 180 determines that the vital sign of the user is weak (e.g., the vital sign is not within a safety range), the alarm module 130 may trigger the emergency medical alert, so that medical personnel and family members may understand the situation and seize a critical rescue window.

The religious music playback module 190 is configured to play the religious music. In this embodiment, if the vital detection module 180 determines that the vital sign of the user is weak or that the user is in a state of imminent death, the religious music playback module 190 initiates the playback of religious music, such as Buddhist scriptures, to provide the user with spiritual comfort before rescue is obtained. The religious music may include, for example, Buddhist scriptures or requiems, and may be selected based on the user's religious beliefs.

FIG. 2 is a block diagram of the personal care device in accordance with another embodiment of the present disclosure.

Compared to FIG. 1, the fall detection module 110 in FIG. 2 includes at least one of a second infrared thermal imaging detection module 112, a millimeter wave detection module 114, and an image vision detection module 116.

The second infrared thermal imaging detection module 112 generates a second infrared thermal imaging signal. The second infrared thermal imaging detection module 112 is an infrared sensor with a wide field of view (e.g., greater than 60 degrees) and high resolution (e.g., greater than 60×80 pixels), and is used to detect images with a wider field of view or higher resolution.

The second infrared thermal imaging signal includes information pertaining to temperature, breathing, and heartbeat or heart rhythm, and represents a signal with a wider field of view or higher resolution. The temperature, breathing, and heartbeat or heart rhythm may be utilized to determine the physical condition of the user, for example, whether the body temperature is within a normal range, whether the breathing is within a normal frequency range, or whether the heartbeat or heart rhythm is within a normal frequency range.

In comparison to the first infrared thermal imaging detection module 160, the second infrared thermal imaging detection module 112 detects images with a wider field of view or higher resolution, which requires a greater expenditure of image processing resources.

The millimeter wave detection module 114 emits, receives, and processes a millimeter-wave signal, and calculates the movement distance of the user, which is used by the fall data processing module 125 to determine whether the user has fallen. The millimeter wave detection module 114 may be a millimeter wave radar.

The image vision detection module 116 senses images of the user within the living environment. The image vision detection module 116 may be a camera or a module with an independent image processor.

The one or more types of activity signals include thermal image signals, millimeter-wave signals, and color image signals.

It is worth mentioning that the signals generated by the second infrared thermal imaging detection module 112 have a larger range or higher resolution compared to the signals generated by the first infrared thermal imaging detection module 160, which may cause the fall data processing module 125 to make the false determination in the first fall detection alert. Furthermore, the millimeter wave detection module 114 calculates distance and may also cause the fall data processing module 125 to make the false determination. The image vision detection module 116 provides color image signals, and the fall data processing module 125 requires a higher computational cost. Furthermore, the color image data may compromise the user's privacy, and the user may experience a negative feeling of being monitored. The fall detection module 110 may not include the image vision detection module 116 to protect the user's privacy.

In addition, the determination mechanism for the first fall detection alert may result in the false determination of the user's actions due to obstructions. Therefore, in order to avoid the false determination in the first fall detection alert that could lead to unnecessary erroneous notifications, the personal care device of the present disclosure, after triggering the user fall detection alarm, does not directly determine that the user has fallen, but instead further confirms the second fall detection alert. In the second fall detection alert, the first infrared thermal imaging signal is a low-range or low-resolution thermal image signal. The thermal image signal may still be analyzed to determine the body temperature, breathing, and heartbeat or heart rate of the user. However, due to the smaller range or lower resolution, the data volume is low, allowing for a rapid determination of whether the body temperature, breathing, heartbeat, or heart rate of the user is normal. For example, in the second fall detection alert, the vital detection module 180 determines the user's condition by determining whether at least one of the body temperature, breathing, heartbeat, or heart rate of the user is outside the normal range (this unfavorable condition is often caused by the user falling) and requires assistance from others. In the second fall detection alert, the alarm module 130 triggers the emergency medical alert, indicating that the user is in a very urgent and dangerous situation and requires medical personnel to expedite assistance to the user.

With this double-check mechanism, the personal care device may further confirm the user's status in the second fall detection alert, significantly reducing the problem of false determination. Because the personal care device eliminates the possibility of false determination of the first fall detection alert, the embarrassing situation where the religious music playback module 190 plays the religious music while the user is still alive does not occur. Therefore, when the vital detection module 180 confirms that the vital sign of the user are poor (e.g., near death or deceased) and medical personnel are unable to provide assistance, the religious music playback module 190 initiates the playback of religious music, which may assist the user who is in poor condition to calmly face death nearing the end of life and guide the soul into the afterlife, demonstrating a high degree of humanistic concern.

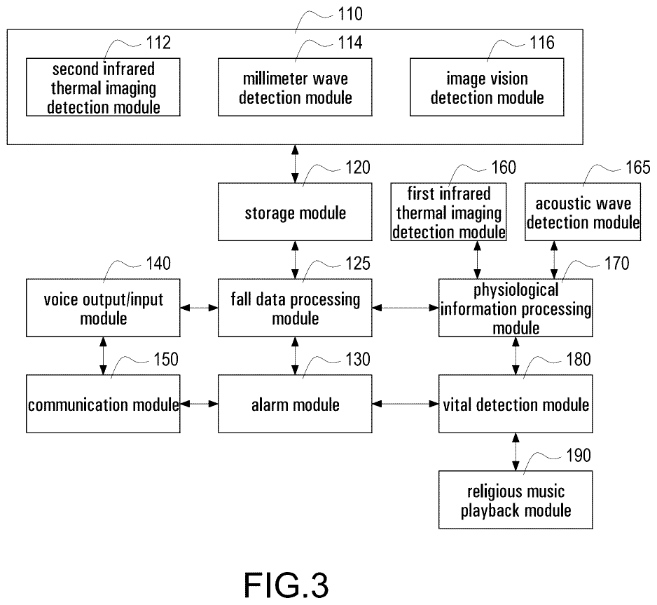

FIG. 3 is a block diagram of the personal care device in accordance with still another embodiment of the present disclosure.

In comparison to FIG. 2, the personal care device further includes an acoustic wave detection module 165.

The acoustic wave detection module 165 is coupled to the physiological information processing module 170. The acoustic wave detection module 165 is configured to detect a user's acoustic wave and to compare the user's acoustic wave with an abnormal physiological acoustic sample. The abnormal physiological acoustic sample may include pre-stored sounds such as groans, cries of pain, or vocalizations commonly associated with falls. The acoustic wave detection module 165, by comparing the user's acoustic wave with the sound characteristics of the abnormal physiological acoustic sample, may determine the current physical state of the user.

The fall detection module 110 includes, for example, but is not limited to, a processor configured to execute fall detection.

The storage module 120 includes, for example, but is not limited to, a random access memory (RAM), a flash memory, read-only memory (ROM), a hard disk drive (HDD), a solid-state drive (SSD), an optical storage device, or a combination of the aforementioned components.

The fall data processing module 125 includes, for example, but is not limited to, a processor configured to execute fall data analysis.

The alarm module 130 includes, for example, but is not limited to, a speaker and an audio playback device, or an audio/video playback device, and may be integrated with the voice output/input module 140 as a single module.

The voice output/input module 140 includes, for example, but is not limited to, a speaker and an audio playback device, or an audio/video playback device.

The communication module 150 includes, for example, but is not limited to, a communication chip that supports Global System for Mobile communication (GSM), Long Term Evolution (LTE), fifth generation (5G) networks, or any subsequent network systems, Wireless Fidelity (Wi-Fi), Bluetooth technology, or wired networks, or a combination of the aforementioned components.

The acoustic wave detection module 165 includes, for example, but is not limited to, a processor configured to perform an acoustic wave detection.

The physiological information processing module 170 includes, for example, but is not limited to, a processor configured to perform physiological information analysis.

The vital detection module 180 includes, for example, but is not limited to, a processor configured to perform vital sign detection analysis.

The religious music playback module 190 includes, for example, but is not limited to, an audio player.

The fall detection module 110, the fall data processing module 125, the acoustic wave detection module 165, and the vital detection module 180 may be integrated into a single processor or processor array, and each may execute the functions described above.

The personal care device and the electronic device or remote control center communicate therewith are equipped with an application; the personal care device and the electronic device or remote control center, in conjunction with the application, may execute the rescue monitoring and religious music playback method of the present disclosure.

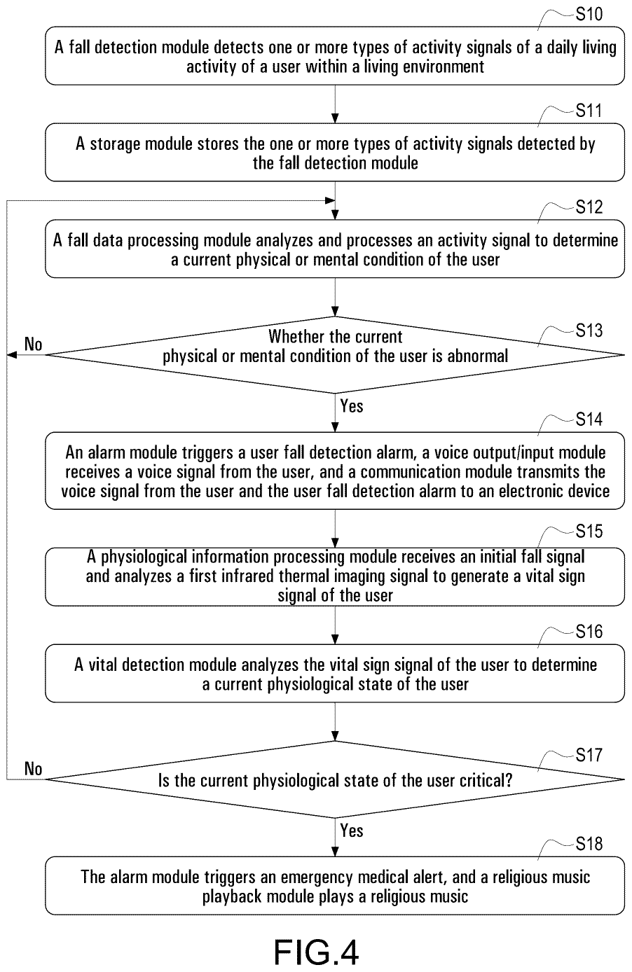

FIG. 4 is a flowchart of the rescue monitoring and religious music playback method in accordance with the present disclosure. The rescue monitoring and religious music playback method may be executed by the personal care device as shown in FIGS. 1 through 3.

In step S10, the fall detection module 110 detects one or more types of activity signals of the daily living activity of the user within the living environment.

In step S11, the storage module 120 stores the one or more types of activity signals detected by the fall detection module 110.

In step S12, the fall data processing module 125 analyzes and processes the activity signal to determine a current physical or mental condition of the user.

In step S13, the fall data processing module 125 determines whether the current physical or mental condition of the user is abnormal; if abnormal, step S14 is executed; if normal, the process returns to step S12.

In step S14, the alarm module 130 triggers a user falls detection alarm, the voice output/input module 140 receives a voice signal from the user, and the communication module 150 transmits the voice signal from the user and the user fall detection alarm to an electronic device.

In step S15, the physiological information processing module 170 receives the initial fall signal and analyzes the first infrared thermal imaging signal to generate a vital sign signal of the user.

In step S16, the vital detection module 180 analyzes the vital sign signal of the user to determine the current physiological state of the user.

In step S17, if the current physiological state of the user is critical, then step S18 is executed; otherwise, it indicates that step S14 was the false determination, and the process returns to step S12 to continue monitoring.

In step S18, because the current physiological state of the user is critical, the alarm module 130 triggers an emergency medical alert, and the religious music playback module 190 plays the religious music, providing the user with spiritual comfort before rescue is obtained after a fall.

In summary, the personal care device and the rescue monitoring and religious music playback method of the present disclosure utilize a plurality of types of sensor modules to perform non-contact detection of the monitoring environment. Through the detection of the fall and the physiological state of the user, and through real-time voice communication, the user's condition is monitored and confirmed. If the user is in a life-threatening or deceased state, medical personnel or family members are promptly notified to provide emergency assistance. Furthermore, if the user does not receive immediate assistance, religious music playback may provide spiritual comfort.

The foregoing description constitutes only a specific example of the present disclosure and is not intended to limit the scope of the claims. Therefore, any equivalent variations made by utilizing the content of the present disclosure are likewise included within the scope of the present disclosure, and it is hereby expressly stated.

What is claimed is:

1. A personal care device, comprising:
a fall detection module, configured to detect one or more types of activity signals of a daily living activity of a user within a living environment;
a fall data processing module, configured to analyze and process the one or more types of activity signals and to generate an initial fall signal when determining that the user has fallen;
an alarm module, coupled to the fall data processing module, and configured to trigger a user fall detection alarm based on the initial fall signal;
a first infrared thermal imaging detection module, configured to generate a first infrared thermal imaging signal associated with the daily living activity of the user;
a physiological information processing module, coupled to the first infrared thermal imaging detection module and the fall data processing module, and configured to analyze the first infrared thermal imaging signal based on the initial fall signal to generate a vital sign signal of the user;
a vital detection module, coupled to the physiological information processing module and the alarm module, and configured to analyze the vital sign signal of the user to determine a current physiological state of the user; and
a religious music playback module, coupled to the vital detection module, wherein when the vital detection module determines that a vital sign of the user is outside a safety range, the alarm module triggers an emergency medical alert, and the religious music playback module plays a religious music within the living environment of the user for the user to listen to.

2. The personal care device of claim 1, further comprising:
an acoustic wave detection module, coupled to the physiological information processing module, configured to detect an acoustic wave of the user and to compare the acoustic wave of the user with an abnormal physiological acoustic sample to determine a current physical state of the user, and to transmit the current physical state of the user to the vital detection module;
wherein the vital detection module generates the vital sign signal of the user based on at least one of the first infrared thermal imaging signal and the current physical state of the user generated by the acoustic wave detection module.

3. The personal care device of claim 1, wherein the fall detection module comprises at least one of a second infrared thermal imaging detection module, a millimeter wave detection module, and an image vision detection module, wherein the second infrared thermal imaging detection module is configured to generate a second infrared thermal imaging signal, the millimeter wave detection module is configured to emit, receive, and process a millimeter-wave signal to calculate a movement distance of the user, and the image vision detection module is configured to sense an image of the user within the living environment.

4. The personal care device of claim 3, wherein the first infrared thermal imaging detection module is an infrared thermal imaging sensor with a field of view less than or equal to 60 degrees and/or a resolution less than or equal to 60×80 pixels, and the second infrared thermal imaging detection module is an infrared thermal imaging sensor with a field of view greater than 60 degrees and/or a resolution greater than 60×80 pixels.

5. The personal care device of claim 1, further comprising:
a storage module, coupled to the fall detection module and the fall data processing module, and configured to store the one or more types of activity signals;
a voice output/input module, coupled to the fall data processing module and configured to receive a voice signal from the user and to play a remote voice signal from an electronic device; and
a communication module, coupled to the voice output/input module and configured to establish remote voice communication with the electronic device.

6. A rescue monitoring and religious music playback method, adapted for execution by a personal care device, the personal care device comprising a fall detection module, a fall data processing module, an alarm module, a physiological information processing module, a vital detection module, and a religious music playback module, and the rescue monitoring and religious music playback method comprising:
detecting one or more types of activity signals of a daily living activity of a user within a living environment through the fall detection module;
analyzing and processing the one or more types of activity signals and generating an initial fall signal when the fall data processing module determines that the user has fallen through the fall data processing module;
triggering a user fall detection alarm based on the initial fall signal through the alarm module;
receiving the initial fall signal and analyzing a first infrared thermal imaging signal generated by a first infrared thermal imaging detection module to generate a vital sign signal of the user through the physiological information processing module;
analyzing the vital sign signal of the user to determine a current physiological state of the user through the vital detection module; and
when the vital detection module determines that a vital sign of the user is not within a safety range, triggering an emergency medical alert through the alarm module and playing religious music in the living environment of the user for the user to listen to through the religious music playback module.

7. The rescue monitoring and religious music playback method of claim 6, wherein the personal care device comprises an acoustic wave detection module coupled to the physiological information processing module, and wherein the rescue monitoring and religious music playback method further comprises:
detecting an acoustic wave of the user and comparing the acoustic wave of the user with an abnormal physiological acoustic sample to determine the current physical state of the user, and transmitting the current physical state of the user to the vital detection module through the acoustic wave detection module.

8. The rescue monitoring and religious music playback method of claim 7, further comprising:
generating the vital sign signal of the user based on at least one of the first infrared thermal imaging signal and the current physical state of the user generated by the acoustic wave detection module through the vital detection module.

9. The rescue monitoring and religious music playback method of claim 6, wherein the personal care device comprises a storage module, a voice output/input module, and a communication module, and the rescue monitoring and religious music playback method further comprises:
storing the one or more types of activity signals through the storage module;
receiving a voice signal from the user and playing a remote voice signal from an electronic device through the voice output/input module; and
establishing remote voice communication with the electronic device through the communication module.

10. The rescue monitoring and religious music playback method of claim 6, further comprising:
when the vital detection module determines that the vital sign of the user is outside the safety range, triggering the emergency medical alert to an electronic device through the alarm module, such that the electronic device emits an emergency distress message.
